(19) United States
(12) Patent Application Publication
(10) Pub. No.: US 2025/0169833 A1
(43) Pub. Date: May 29, 2025

(54) SYSTEMS AND METHODS FOR PULMONARY EMBOLI REMOVAL

(71) Applicant: SCIENTIA VASCULAR, INC., West Valley City, UT (US)

(72) Inventors: John A. LIPPERT, Park City, UT (US); William Rigby ECCLES, Centerville, UT (US); Brandon Rodney POPE, Salt Lake City, UT (US)

(21) Appl. No.: 18/842,983

(22) PCT Filed: Mar. 9, 2023
(86) PCT No.: PCT/US2023/064076

§ 371 Date: Aug. 30, 2024

Related U.S. Application Data
(60) Provisional application No. 63/318,490, filed on Mar. 10, 2022.

(51) Int. Cl. A61B 17/22; A61B 17/00

(52) U.S. Cl. CPC A61B 17/22; A61B 2017/00557; A61B 2017/22067; A61B 2017/22079; A61B 2217/005

(57) ABSTRACT

A system for treating pulmonary embolism includes an aspiration catheter having a distal occluding component and a holding catheter having a distal valve accessory. In use, expansion of the occluding component blocks a target pulmonary artery, and deployment of the valve accessory promotes mitral valve regurgitation. Suction applied by the aspiration catheter induces a reversal of blood flow throughout a targeted pulmonary region. Reversing the direction of blood flow enables removal of one or more pulmonary emboli.

### CROSS-REFERENCE TO RELATED APPLICATION

This application claims priority to and the benefit of U.S. Provisional Patent Application No. 63/318,490, filed Mar. 10, 2022, and titled “Systems and Methods for Pulmonary Emboli Removal,” which is incorporated herein by reference in its entirety.

### BACKGROUND

### Technical Field

This disclosure relates to catheter-based devices, systems, and methods of use thereof, for use in removal of pulmonary emboli.

### Related Technology

Acute pulmonary embolism is a significant cause of mortality worldwide, with over 100,000 deaths per year in the U.S. alone. An acute pulmonary embolism (PE), or embolus, is a blockage of a pulmonary (lung) artery. Most often, the condition results from a blood clot that forms in the legs or another part of the body (e.g., deep vein thrombosis, or DVT) and travels to the lungs. Following myocardial infarction and stroke, PE is the third leading cardiovascular cause of death. Modern medical treatment of acute PE generally falls within four categories: systemic anticoagulation; catheter-based fibrinolysis; systemic fibrinolysis; and surgical pulmonary embolectomy. Treatments may involve a combination of these therapies.

However, current treatments are limited and can result in significant risks to patients. Anti-coagulation approaches are often ineffective, particularly in complex and/or advanced disease profiles. Systemic fibrinolytics have significant associated risks for bleeding and are often contraindicated, meaning that certain conditions suggest or indicate that that particular technique should not be used. Surgical and catheter-based methods are limited by vessel and/or catheter size and by the limited navigability of present catheter technology, particularly through tortuous environments such as the human lung structures.
There is thus a long felt and unmet need for techniques, devices, and systems capable of addressing these and other issues in the art.

### BRIEF SUMMARY

The present disclosure relates to catheter-based systems, devices, and methods for removal of pulmonary emboli. Certain embodiments of disclosed systems, devices, and methods temporarily reverse blood flow within the pulmonary vasculature, facilitating the dislodgement of entrapped emboli. Beneficially, the disclosed systems, devices, and methods enable the effective removal of pulmonary emboli without necessarily requiring the administration of systemic therapeutic agents (or enable a reduction in the use of such agents) and without requiring catheter navigation through small pulmonary vessels. Thus, the disclosed systems, devices, and methods enable safer and more effective treatment of PE.

In some embodiments, a system includes an aspiration catheter with a distal end balloon and a second catheter for holding the mitral valve open during systole. In some embodiments, the second catheter may include a distal accessory to enable holding the mitral valve open. In some embodiments, the distal end balloon of the aspiration catheter may occlude either the right or the left pulmonary artery or subsequent pulmonary arterial vessels.

An embodiment of a disclosed method includes delivering a catheter-based component through the heart via the venous system to access the pulmonary vasculature. The method may also include: occluding a target pulmonary arterial vessel to block normal blood flow; providing suction to allow for reverse flow, emboli removal and/or blood drainage; and optionally administering local pharmaceutical agents into the pulmonary vasculature. Administration of pharmaceutical agents may include, for example, administration of anti-coagulation, anti-inflammatory, anti-platelet, and/or other therapeutic agents. The method may further include delivering a catheter-based component to the mitral valve via the arterial system to prevent the mitral valve from closing during ventricular systole, thus allowing for increased backpressure to reverse pulmonary blood flow.

Using reversed pulmonary blood flow to remove emboli from vessels has been used as a neuroprotective procedure in the treatment of stroke, which also involves the formation of clots or emboli. Here, creating reverse flow across the pulmonary vasculature can be an effective and safe way to treat PE.

To avoid major cardiac disruption, blood flow reversal is preferably limited to a single lung or region of lung tissue within a single lung. A device may be used to occlude blood flow in a target pulmonary arterial vessel to initiate blood flow reversal within a targeted pulmonary region. An external pump may be used to create the negative pressure necessary to reverse flow and to drain retrograde blood and/or particulates. This aspired blood may travel through an inline filter disposed inside an aspiration catheter to remove dislodged emboli before entering the low-pressure venous system, or it may be externally drained, assuming hemodynamic stability (stable blood flow and good circulation).

Once normal blood flow is halted on the pulmonary arterial side, and negative pressure and/or reverse flow drainage is established, more pressure may be required to establish and maintain the reverse flow. To apply more pressure, a component to prevent the mitral valve from closing may be inserted into the heart during normal cardiac rhythm, thus causing pressure to be transmitted retrogradely into the left atrium during ventricular systole, through the desired pulmonary vein, and into the target tissue lung. Keeping the mitral valve open thus facilitates an increase in mitral regurgitation, which further contributes to maintaining the reversal of blood flow.

Additional features and advantages will be set forth in part in the description that follows, and in part will be obvious from the description, or may be learned by practice of the embodiments disclosed herein. The objects and advantages of the embodiments disclosed herein will be realized and attained by means of the elements and combinations particularly pointed out in the appended claims. It is to be understood that both the foregoing brief summary and the following detailed description are exemplary and explanatory only and are not restrictive of the embodiments disclosed herein or as claimed.

### BRIEF DESCRIPTION OF THE DRAWINGS

A more particular description of the invention briefly described above will be rendered by reference to specific embodiments which are illustrated in the appended drawings. Understanding that these drawings depict only typical embodiments of the invention and are not limiting of its scope, the invention will be described and explained with additional specificity and detail through the use of the accompanying drawings in which:

FIGS. 1A-D illustrate a cross-sectional anatomical overview of blood flow with one embodiment of the disclosed reverse flow system, illustrating only one pulmonary artery and vein.

FIGS. 2A-D illustrate a schematic overview of blood flow according to one embodiment of the disclosed reverse flow system.

FIG. 3 illustrates a catheter-based system for pulmonary emboli removal to be used in a reverse flow system.

FIG. 4 illustrates a method of using the catheter-based system such as illustrated in FIG. 3 to reverse blood flow through pulmonary tissue and promote removal of one or more pulmonary emboli.

### DETAILED DESCRIPTION

The present disclosure related to catheter-based systems, devices, and methods for pulmonary emboli removal. For example, embodiments of disclosed systems, devices, and methods temporarily reverse blood flow within the pulmonary vasculature, facilitating the dislodgement of entrapped emboli. Beneficially, the disclosed systems, devices, and methods enable the effective removal of pulmonary emboli without requiring administration of systemic therapeutic agents and without requiring catheter navigation through small pulmonary vessels.

In some embodiments, a system includes an aspiration catheter (also referred to herein as a first catheter, first catheter-based device, or aspiration catheter-based device) with an occluding component such as a balloon (e.g., disposed at or near a distal end) and a holding catheter (also referred to herein as a second catheter, second catheter-based device, or holding catheter-based device) for holding the mitral valve open during systole or at least temporarily disrupting mitral valve function to increase mitral valve regurgitation.

In some embodiments, the second catheter may include a valve accessory to enable engagement with the mitral valve. In some embodiments, the balloon or other occluding component of the aspiration catheter is used to occlude a pulmonary arterial vessel. For example, the balloon of the aspiration catheter may be deployed to occlude either the right or the left pulmonary artery.

An embodiment of a disclosed method includes the steps of: delivering an aspiration catheter to the heart via the venous system to access the pulmonary vasculature; occluding a target pulmonary arterial vessel to block normal blood flow; providing suction via the aspiration catheter to enable reverse flow and emboli removal; and optionally administering local pharmaceutical agents into the pulmonary vasculature. For example, anti-coagulation, anti-inflammatory, anti-platelet and/or other therapeutic agents may be administered and delivered locally. The method may further include delivering a catheter-based component through the heart via the arterial system to prevent the mitral valve from fully closing during ventricular systole, thus allowing for increased backpressure to reverse pulmonary blood flow.

To avoid major cardiac disruption, blood flow reversal is preferably limited to a single lung or region of lung tissue within a single lung. A device may be used to occlude blood flow in a target pulmonary arterial vessel to initiate blood flow reversal. An external pump may be used to create a negative pressure for reverse flow and to drain retrograde blood and/or particulates. This aspired blood may travel through a filter such as an inline filter to remove dislodged emboli before entering the low-pressure venous system, or it can be externally drained.

Once normal blood flow is halted on the pulmonary arterial side and negative pressure/reverse flow drainage is established, more pressure modulation may be required to establish reverse flow. Accordingly, a component to prevent the mitral valve from fully closing may be utilized to cause pressure to be transmitted retrogradely into the left atrium, assisting with blood flow reversal.

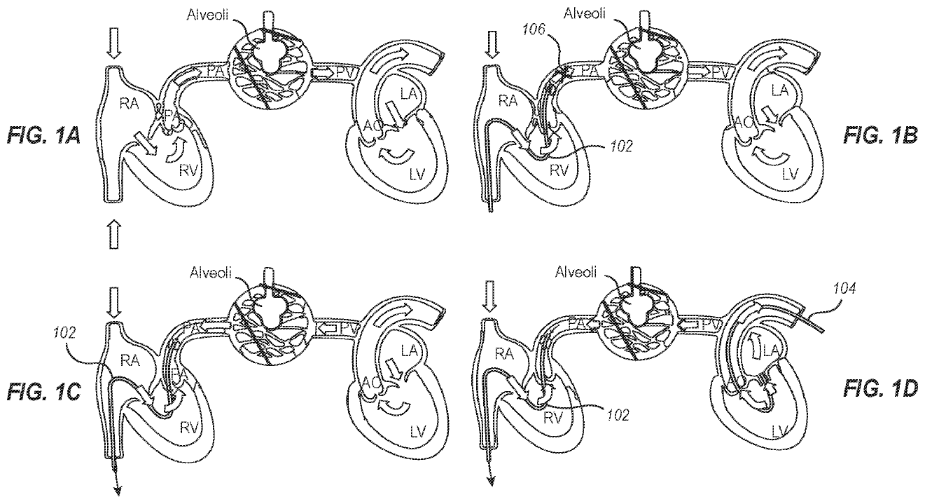

FIGS. 1A-D illustrate a cross-sectional anatomical overview of blood flow with one embodiment of the disclosed reverse flow system, illustrating only one pulmonary artery and vein. It is to be understood that the reverse flow system could be implemented in more than one pulmonary artery and vein.

FIG. 1A illustrates normal blood flow through the heart and lungs, where de-oxygenated blood flows from the right atrium (RA) and right ventricle (RV), through the pulmonary arteries (PA) to the lungs and alveoli where it is oxygenated, through the pulmonary veins (PV) to the left atrium (LA) and left ventricle (LV), and then through the aorta (AO) for distribution throughout the body.

De-oxygenated blood enters the right atrium of the heart from the venous system. The de-oxygenated blood then passes through the tricuspid valve to the right ventricle of the heart, then through the depicted pulmonary artery and into the lung structure (e.g., to the alveoli-capillary bed) where the blood is oxygenated. Freshly oxygenated blood leaves the lungs through the pulmonary veins to enter the left atrium of the heart. The oxygenated blood then travels through the mitral valve and into the left ventricle before being passed into the aorta to be delivered throughout the body.

As illustrated in FIG. 1B, a first catheter-based device 102 may be inserted into the right atrium (e.g., via a transfemoral approach, transradial approach, or other suitable approach), through the right ventricle and into a target pulmonary artery. Deploying an occluding component 106 (e.g., a balloon) in the target pulmonary artery disrupts flow through that pulmonary vessel and its associated downstream pulmonary vasculature.

FIG. 1C illustrates use of the first catheter-based device 102 to provide suction to reverse the flow of blood through the target pulmonary artery. The negative pressure created by the suction from the catheter-based device induces reversal of blood flow. In alterative embodiments, a separate aspiration catheter, distinct from the first catheter 102, can be routed to an area near where the occluding component 106 has been deployed (e.g., such that the distal end is just distal of the occluding component 106) and utilized to provide suction.

FIG. 1D further illustrates a second catheter-based device 104 being inserted into the left ventricle (e.g., via a transfemoral approach, transradial approach, transseptal approach, or other suitable approach) and then into the mitral valve. The second catheter-based device 104 may be deployed to keep the mitral valve open or to at least increase the level of regurgitation. Combined with the negative pressure and suction from the pulmonary arterial side, keeping the mitral valve open facilitates mitral regurgitation and further enables the reversal of blood flow (that is, the flow of blood from the left side of the heart to the right side of the heart).

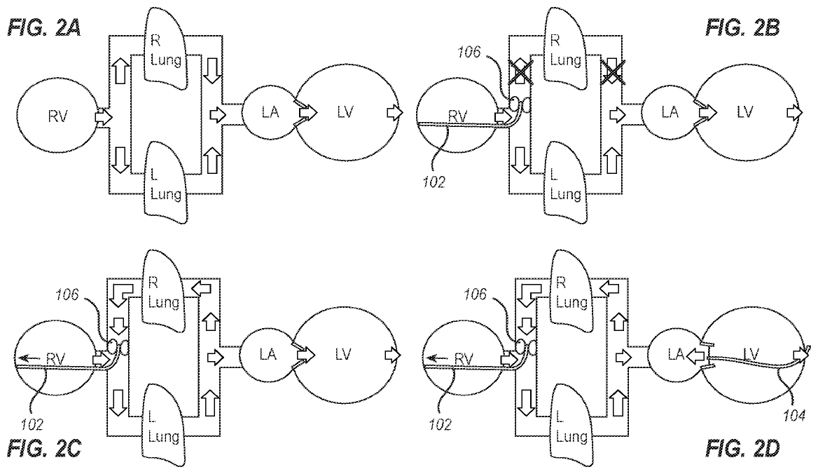

FIGS. 2A-D illustrate a schematic overview of blood flow according to one embodiment of the disclosed reverse flow system. FIG. 2A illustrates normal blood flow through the heart and lungs. De-oxygenated blood enters the right side of the heart from the venous system. The de-oxygenated blood travels through the right ventricle (RV) of the heart, through the right and left pulmonary arteries and into the lung structure (e.g., through to the alveoli in both the right and left lung) where the blood is oxygenated. Freshly oxygenated blood leaves the lungs through the right and left pulmonary veins, entering the left atrium (LA) of the heart. The oxygenated blood then travels through the left atrium to the left ventricle (LV) via the mitral valve, and through the aorta to be delivered throughout the body.

As illustrated in FIG. 2B, the first catheter-based device 102 may be inserted into the right ventricle and may be deployed (e.g., by deploying occluding component 106) to block the right pulmonary artery. Blocking the right pulmonary artery disrupts the normal flow of blood to the right lung. As shown, blood is still flowing normally through the left lung.

FIG. 2C illustrates the first catheter-based device 102 providing suction to reverse the flow of blood through the right pulmonary artery, while still blocking the normal flow of blood through the right pulmonary artery. The negative pressure created by the suction from the first catheter-based device 102 induces reversal of blood flow. As shown, blood is still flowing normally through the left lung.

FIG. 2D illustrates a second catheter-based device 104 being inserted into the left ventricle to increase mitral valve regurgitation. Combined with negative pressure and suction via the first catheter-based device 102 shown in FIGS. 2A-2C, keeping the mitral valve open facilitates mitral regurgitation, which increases backpressure within the target pulmonary vasculature throughout ventricular systole, and further enables the reversal of blood flow through the right lung. As shown, blood is still flowing normally through the left lung.

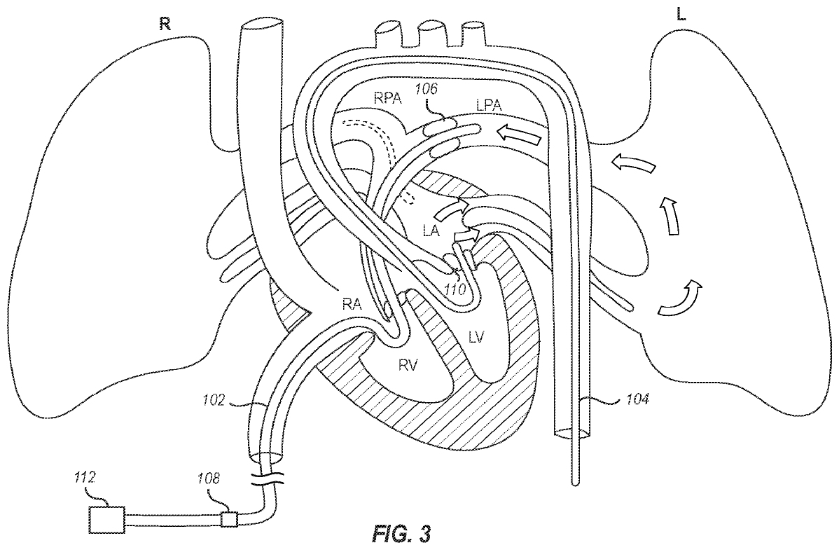

FIG. 3 illustrates a catheter-based system for pulmonary emboli removal to be used in a reverse flow system. The reverse flow system may include an aspiration catheter 102 with an occluding component 106 (e.g., a balloon) and a holding catheter 104. The aspiration catheter 102 may be placed in either the right pulmonary artery (RPA) or left pulmonary artery (LPA) and the occluding component 106 may occlude either the right or left pulmonary arteries upon expansion.

The aspiration catheter 102 may include an inline filter 108 and may be routed to the right side of the heart through, for example, a femoral vein. Blood may flow through the aspiration catheter 102 and the inline filter 108 via the normal physiological pressure gradient or through an external pump 112, such as a mechanical pump. One or more external filters may additionally or alternatively be used.

As shown, the holding catheter 104 may be routed through the aorta to the left ventricle (or alternatively routed to the left ventricle via a transseptal approach), where a valve accessory 110 may be deployed to hold the mitral valve open or to at least disrupt normal function of the mitral valve to increase regurgitation. The holding catheter 104 may include, for example, a selectively retractable stent-based device, balloon, or other expansion element configured to limit mitral valve closure. Beneficially, by limiting mitral valve closure, the holding catheter 104 enables facilitation of mitral valve regurgitation, better allowing for the reversal of blood flow. The facilitation of mitral valve regurgitation increases left atrial pressure during ventricular systole. Increasing left atrial pressure in conjunction with mitral regurgitation and reverse blood flow through the aspiration catheter can beneficially dislodge one or more pulmonary emboli.

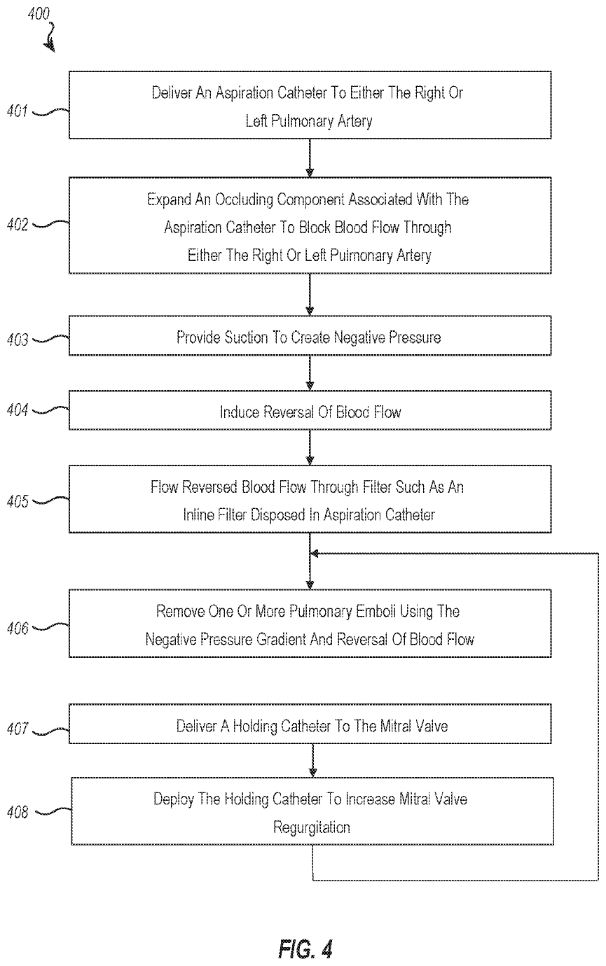

FIG. 4 illustrates a method 400 of using the catheter-based system such as the system illustrated in FIG. 3 to treat a patient suffering from PE. The method may include, for example, delivering an aspiration catheter to either the right or left pulmonary artery (step 401). The method may also include, at step 402, expanding an occluding component (e.g., a balloon) associated with (e.g., connected to the distal end of) the aspiration catheter. In some embodiments, deployment of the occluding device blocks blood flow through whichever of the right or left pulmonary artery was targeted.

Alternatively, the aspiration catheter may be routed further into the pulmonary artery system prior to deployment of the occluding component to target a particular subregion of a lung. For example, the aspiration catheter may be routed into a target lobar artery or segmental artery to target a particular lung lobe or lung segment for reverse blood flow and emboli removal.

The method 400 may further include, at step 403, providing suction through the aspiration catheter to create a negative pressure gradient and, at step 404, inducing a reversal of blood flow. The method may include, at step 405, flowing the reverse blood flow through a filter (e.g., an inline filter disposed within the aspiration catheter) and optionally back into the venous system of the patient. The method may also include, at step 406, removing one or more pulmonary emboli using the negative pressure gradient and reversal of blood flow, thereby treating acute PE in a patient.

The method 400 may further include, at step 407, delivering a holding catheter to the mitral valve and, at step 408, deploying the holding catheter to block full closure of the mitral valve and/or otherwise increase mitral valve regurgitation. Steps 407 and 408 may be performed concurrently with any of steps 401-406.

### Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, numbers expressing quantities, constituents, distances, or other measurements used in the specification and claims may optionally be modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

1. A system for treating pulmonary emboli, comprising:
an aspiration catheter comprising an occluding component attached at or near a distal end of the aspiration catheter; and
a holding catheter comprising a valve accessory attached at or near a distal end of the holding catheter,
wherein the occluding component is configured to block normal blood flow through one or more pulmonary arterial vessels upon expansion.

2. The system of claim 1, wherein the valve accessory comprises a stent-based device or a balloon.

3. The system of claim 1, further comprising a suction device configured to provide suction to the one or more pulmonary arteries through the aspiration catheter to induce a reversal of blood flow through the aspiration catheter.

4. The system of claim 3, wherein the suction device comprises an external pump.

5. The system of claim 1, wherein the occluding component comprises a balloon.

6. The system of claim 1, further comprising an inline filter disposed within the aspiration catheter.

7. A method of treating pulmonary embolism, the method comprising:
delivering a first catheter to a target pulmonary arterial vessel, wherein the first catheter comprises an occluding component disposed at or near a distal end;
deploying the occluding component; and
providing suction to the target pulmonary arterial vessel to induce a reversal of blood flow.

8. The method of claim 7, further comprising dislodging one or more pulmonary emboli.

9. The method of claim 8, further comprising removing one or more pulmonary emboli from the patient.

10. The method of claim 7, wherein providing suction to the target pulmonary arterial vessel comprises delivering an aspiration catheter, distinct from the first catheter, to the target pulmonary arterial vessel to provide suction.

11. The method of claim 7, wherein the step of providing suction to the target pulmonary arterial vessel comprises providing suction through the first catheter.

12. The method of claim 7, wherein the method treats acute pulmonary embolism.

13. The method of claim 7, wherein the occluding component comprises a selectively expandable balloon.

14. The method of claim 7, further comprising:
providing a holding catheter, wherein the holding catheter comprises a valve accessory at or near a distal end; and
deploying the valve accessory of the holding catheter at or near the mitral valve to increase mitral regurgitation.

15. The method of claim 14, wherein increasing mitral regurgitation further induces the reversal of blood flow.

16. The method of claim 7, further comprising flowing the reversed blood flow through a filter.

17. The method of claim 16, wherein the filter is an inline filter disposed in the first catheter.

18. The method of claim 16, further comprising returning the reversed blood flow to the venous system of the patient.

19. A method of treating pulmonary embolism, the method comprising:
delivering a first catheter to a target pulmonary arterial vessel, wherein the first catheter is an aspiration catheter, and wherein the first catheter comprises an occluding component disposed at or near a distal end;
deploying the occluding component within the target pulmonary arterial vessel;
providing a holding catheter, wherein the holding catheter comprises a valve accessory at or near a distal end;
deploying the valve accessory of the holding catheter at or near the mitral valve to increase mitral regurgitation; and
providing suction to the target pulmonary arterial vessel by way of the first catheter to induce a reversal of blood flow.

20. (canceled)
